(19) United States
(12) Patent Application Publication
(10) Pub. No.: US 2015/0056170 A1
(43) Pub. Date: Feb. 26, 2015

(54) PLACENTAL STEM CELLS, METHODS FOR ISOLATING SAME AND USE THEREOF

(71) Applicant: SCT&B INC., Sherbrooke (CA)

(72) Inventor: Aziz Aris, Sherbrooke (CA)

(21) Appl. No.: 14/382,243

(22) PCT Filed: Mar. 6, 2013
(86) PCT No.: PCT/CA2013/050167

§ 371 Date: Aug. 29, 2014

Related U.S. Application Data
(60) Provisional application No. 61/607,150, filed on Mar. 6, 2012.

(51) Int. Cl. A61K 35/50; G01N 33/569; C12N 5/0735

(52) U.S. Cl. CPC A61K 35/50; C12N 5/0606; G01N 33/56966
USPC 424/93.7; 435/366; 435/7.21

(57) ABSTRACT

The present description relates to an isolated population of human placental pluripotent stem cells or an isolated human placental pluripotent stem cell, positive for human leucocyte antigen-G (HLA-G), a migration marker and at least one pluripotent stem cell marker. The present description further provides a method for isolating human placental pluripotent stem cells. The method comprising: extracting cells from a human placenta; and isolating cells positive for human leucocyte antigen-G (HLA-G), a migration marker and at least one pluripotent stem cell marker and use thereof. On the other hand, it is likely that this present description is applicable to primates and other animals.

The present description relates to placental embryonic stem cells, methods for isolating same and use thereof.

Regenerative medicine (RM) is an emerging and rapidly evolving field of research and therapeutics. It aims to restore cellular function by repairing, replacing or regenerating cells, tissue or organs (Daar A S et al. A proposed definition of regenerative medicine J Tissue Eng Regen 2007 p. 179-184). Regenerative medicine could ultimately help to provide therapeutic treatment for conditions where current therapies are inadequate. These conditions are numerous and include, among other, diabetes, acute liver and heart failures, muscular disorders, arthritis, brain damages and disorders, vision disorders, renal disorders, and hematopoietic and immune diseases, as well as acute leukemia and lymphoma and diverse solid tumor types. The human body has an endogenous system of regeneration through stem cells. The latter can be found almost in every type of tissue.

Stem cells are self-renewable and can differentiate into various cell lineages. These cells can be classified into embryonic stem cells (ESC) and non-embryonic or adult stem cells (ASC). The latter cannot differentiate into all types of tissues (pluripotent) and proliferate at a lower rate than ESC. Therefore the use of ESC is preferred over the use of ASC.

Preliminary studies in animals and humans have suggested that symptoms of some diseases may be alleviated by transplanting stem cells upon cultivating these cells in vitro such as diabetes, Parkinson's disease, hepatic failure and congestive heart failure. However, this field is a slow evolving field due to many reasons such as ethical issue raised with the use of human ESC and our very limited knowledge of the field.

Due to the drawbacks mentioned above, use of ASC derived from bone marrow in human has been developed to treat certain cancers such as blood cancer (leukemia). However, this therapy is not systematically practiced and remains dependent on the willingness of some clinicians as it requires invasive surgery, extensive expertise and specialized centers. Some researchers are developing cell therapy based on stem cells isolated from umbilical cord. However, the quantity of stem cells that can be extracted from the umbilical cord is relatively low which makes this approach a lengthy and costly process. In addition, stem cells isolated from the umbilical cord are not immunocompatible with every receiver promoting an immune response resulting in graft rejection.

Induced pluripotent stem cells (iPSCs) offer immense potential for regenerative medicine. Lister et al (Hotspots of aberrant epigenomis reprogramming human in human induced pluripotent stem cells, Nature, 471, 2011, p. 68-73) demonstrate that human iPSCs show significant reprogramming variability, including somatic memory and aberrant epigenomic reprogramming.

There is therefore a need for a cell therapy that would allow 1) an ethical recovery of a large quantity of pluripotent stem cells not derived from an embryo; 2) production of immunocompatible stem cells with any receiver; and 3) production of guidable stem cells that would reach a target of interest.

### SUMMARY

The present description provides pluripotent stem cells isolated from a human placenta. The population of stem cells comprises at least one stem cell pluripotent marker, human leucocyte antigen-G (HLA-G), as well as a migration marker. As such, the stem cells population of the present description is immunocompatible with any receiver, can migrate to a target tissue and does not raise an ethical issue as the placenta is discarded following delivery. In addition the placenta allows the recovery of a large quantity of stem cells.

The present description provides an isolated population of human placental pluripotent stem cells or an isolated human placental pluripotent stem cell positive for human leucocyte antigen-G (HLA-G), a migration marker and at least one pluripotent stem cell marker.

The present description further provides a method for isolating human placental pluripotent stem cells, the method comprising:
extracting cells from a human placenta; and
isolating cells positive for human leucocyte antigen-G (HLA-G), a migration
marker and at least one pluripotent stem cell marker.

The present description also provides a use of human placental pluripotent stem cells for therapeutic purposes in regenerative medicine, drug delivery, drug discovery, cell cosmetic or gene therapy wherein the human placental pluripotent stem cells are positive for human leucocyte antigen-G (HLA-G), a migration marker and at least one pluripotent stem cell marker.

The present description also provides a use of human placental pluripotent stem cells obtained by the method as described herein in medical imaging wherein the human placental pluripotent stem cells are positive for human leucocyte antigen-G (HLA-G), a migration marker and at least one pluripotent stem cell marker.

The present description also provides a use of human placental pluripotent stem cells as a model for human genetic disorders or as a model for toxicity testing, wherein the human placental pluripotent stem cells are positive for human leucocyte antigen-G (HLA-G), a migration marker and at least one pluripotent stem cell marker.

The present description further provides a cell therapy method for treating a patient in need thereof, the method comprising administering to the patient human placental pluripotent stem cells positive for human leucocyte antigen-G (HLA-G), a migration marker and at least one pluripotent stem cell marker.

### BRIEF DESCRIPTION OF THE FIGURES

FIG. 1. MACSorting of SSEA4, HLA-G and CXCR4 cells.

FIG. 2. MACSorting and FACSorting. MACSorting is performed to isolate cells positive for CXCR4 marker and a FACSorting is further performed with the cells positive for CXCR4 to isolate cells positive for SSEA4 and HLA-G.

FIG. 3. MACSorting of SSEA4, HLA-G and CXCR4 cells.

FIG. 4. MACSorting and FACSorting. MACSorting is performed to isolate cell positive for CXCR4 marker and a FACSorting is further performed with the cells positive for CXCR4 to isolate cells positive for SSEA4 and HLA-G.

FIG. 5. MACSorting of SSEA4, NANOG, ALP and OCT-4.

FIG. 6. Cells viability.

FIG. 7. A representative illustration of FACScan analysis confirming the presence of SSEA4 or HLA-G.

FIG. 8. Representative illustration of CXCR4 immunostaining in stem cells of interest.

### DESCRIPTION

The term “human placenta” refers to the organ that connects the developing fetus to the uterine wall to allow nutrient uptake, waste elimination and gas exchange via the mother's blood supply. Upon a delivery, the placenta is normally discarded. Although the placenta could be obtained following a normal baby delivery, it could also be obtained upon an abortion.

The expression “pluripotent stem cell” refers to a cell that has the potential to differentiate into any of the three germ layers: endoderm, mesoderm or ectoderm. Pluripotent stem cells can therefore give rise to any fetal or adult cell type. However, it is known that pluripotent cells cannot by themselves develop into a fetal or adult organism as they lack the potential to contribute to extraembryonic tissue, such as the placenta. The pluripotent stem cells of the present description are isolated from the placenta. Techniques to isolate pluripotent stem cells from the placenta are known such as enzymatic digestion, Fluorescence activated cell sorter (FACS) and Magnetic activated cell sorter (MACS) for cells suspended in a stream of fluid.

The expression “positive for” refers to the cell expressing a marker. In one aspect, the marker is expressed at the surface of the cell. Techniques for detecting cells positive for a marker are known such as techniques relying on antibodies capable of binding to a selected marker. For example a first antibody specific for a marker is incubated with the cells. The antibody binds to the specific marker and a second labelled antibody specific for the first antibody is incubated with the cells. As such, if the cells express the marker, then the cells are labelled and could be detected. If the cells do not express the marker, no labelling is detected. Methods such as FACSorting, MACSorting or ELISA could be used.

The expression “human leucocyte antigen-G” (HLA-G) refers to a polypeptide known to be encoded by the HLA-G gene. This protein belongs to the HLA nonclassical class I heavy chain paralogues. The class I molecule is a heterodimer consisting of a heavy chain and a light chain (beta-2 microglobulin). The heavy chain is anchored in the membrane. HLA-G is known to be expressed on fetal derived placental cells which may play a role in immune tolerance in pregnancy. In addition, cells expressing HLA-G provide an immunotolerable character upon grafting and thus are immunocompatible with any receiver (Le Maux A et al. Soluble human leucocyte antigen-G molecules in peripheral blood haematopoietic stem cell transplantation: a specific role to prevent acute graft-versus-host disease and a link with regulatory T cells. Clin Exp Immunol. 2008 April; 152(1):50-56). As such the pluripotent stem cells of the present description positive for HLA-G, promote immune tolerance upon grafting and thus are immunocompatible with any receiver.

In one embodiment, the HLA-G polypeptide includes a sequence at least 65% to 95% identical, at least 65%, 70%, 75%, 80%, 85%, 90% or 95% identical to part or all of the nucleic acid or amino acid sequence of HLA-G. It will be understood that the polypeptide having a percentage of identity with HLA-G retains the activity of promoting immune tolerance upon grafting. It will also be understood that the polypeptide having a percentage identity with HLA-G retains the antibody binding capacity in order to be detected. Antibodies used to detect cells positive for HLA-G are known such as Ms mAb HLA-G (MEMG/9)-APC from Thermo Scientific.

The expression “migration marker” refers to a polypeptide expressed by the cell allowing the cell to migrate to a target of interest. As such, the cell expressing the migration marker could reach a specific location or target of interest. In one embodiment, the migration marker is chemokine receptor (CXCR) 4, CXCR5, CXCR6, CXCR7, CCR1, CCR2, CCR3, CCR4, CCR7, CCR9, platelet-derived growth factor receptor (PDGF-Rα), PDGF-Rβ, insulin-like growth factor receptor (IGF-R), RANTES-R, or MDC-R. Migration markers that could be used in accordance with the present description include those described in Honczarenko M et al. Stem cells 2006; 24:1030-1041; Si Y et al. J Clin Invest. 2010; 120(4):1192-1203 and Ponte A L, et al. Stem Cells 2007 July; 25(7):1737-45. Epub 2007 Mar. 29. In one embodiment, the migration marker is CXCR4.

Proinflammatory stimuli (such as wounded tissue, lipopolysaccharides, TNF or IL1) induces the production of stromal cell-derived factor 1-alpha and beta (SDF-1) which is known to attract cells expressing CXCR4 receptor and to bind thereto. As such, the present inventor believes that the pluripotent stem cell of the present description positive for a migration marker could migrate toward an inflammation site upon systemic administration. In addition, upon a local administration, the pluripotent stem cell being attracted by the local target stays locally.

In one embodiment, the migration marker includes a sequence at least 65% to 95% identical, at least 65%, 70%, 75%, 80%, 85%, 90% or 95% identical to part or all of the nucleic acid or amino acid sequence of the above mentioned migration marker. It will be understood that the polypeptide having a percentage of identity with the migration marker retains the activity of the migration polypeptide such that said polypeptide could migrate towards a target. It will also be understood that the polypeptide having a percentage of identity with the migration marker retains the antibody binding capacity in order to be detected. Antibodies used to detect cells positive for CXCR4 are known such as Ms mAb CXCR4 from Abcam and Ms mAb CXCR4-PE-Cy™5 from BD Pharmingen.

The expression <<at least one pluripotent stem cell marker>> refers to a polypeptide expressed by stem cell that differentiates it from any other cell type such as totipotent cell, multipotent cell, oligopotent cell, unipotent cell, blood cell, hepatic cell, endothelial cell and so on. As such, the pluripotent stem cell expressing said polypeptide could be isolated from any other cell types. As known, a pluripotent stem cell could be positive for more than one pluripotent stem cell marker.

In one embodiment, the pluripotent stem cell marker is stage-specific embryonic antigen (SSEA)4, SSEA3, POU5F1/OCT4, NANOG, SOX2, alkaline phosphatase (ALP), human embryonic stem cell antigen-1 (HESCA-1), developmental pluripotency associated 5 (DPPA5), forkhead box D3 (GENESIS/FOXD3), undifferentiated embryonic cell transcription factor 1 (UTF1), TRA-1-60, TRA-1-81, DNA (cytosine-5-)-methyltransferase 3 beta (DNMT3B), teratocarcinoma-derived growth factor 1 (TDGF1/CRIPTO), reduced expression gene 1 (REX1/ZFP42), telomerase reverse transcriptase (TERT), ATP-binding cassette sub-family G member 2 (ABCG2), connexin-43, connexin-45, GCTM2, GCT343, thymus cell antigen (Thy1/CD90), gamma-aminobutyric acid receptor subunit beta-3 (GABRB3), CD9, growth differentiation factor-3 (GDF3), STELLAR, or fibroblast growth factor 4 (FGF4). Pluripotent stem cell markers that could be used in accordance with the present description include those described in International Stem Cell Initiative. Nat Biotechnology. 2007; 25:803-816.

In one embodiment, the pluripotent stem cell marker is SSEA4, NANOG, ALP or OCT4. In another embodiment, the pluripotent stem cell is positive for SSEA4, NANOG, ALP and OCT4. In another embodiment, the pluripotent stem cell marker is SSEA4.

In one embodiment, the pluripotent stem cell marker includes a sequence at least 65% to 95% identical, at least 65%, 70%, 75%, 80%, 85%, 90% or 95% identical to part or all of the nucleic acid or amino acid sequence of the above mentioned pluripotent stem cell marker. It will be understood that the polypeptide having a percentage identity with the pluripotent stem cell marker retains the antibody binding capacity in order to be detected. Antibodies used to detect cells positive for pluripotent stem cell marker are known such as Ms mAb Anti h/m SSEA4 from R&D, Ms mAb NANOG (NNG-811), Ms mAb ALP (BGN/03/661) and Ms mAb OCT4 from Abcam.

Techniques for determining nucleic acid and amino acid “sequence identity” are also known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) could be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm could be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Another method of establishing percent identity which could be used in the context of the present description is the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm could be employed where default parameters are used for the scoring table (for example, gap open penalty of 12, gap extension penalty of one, and a gap of six). From the data generated the “Match” value reflects “sequence identity.” Other suitable programs for calculating the percent identity between sequences are generally known in the art, for example, another alignment program is BLAST, used with default parameters. For example, BLASTN and BLASTP could be used using the following default parameters: genetic code=standard; filter=none; strand=both; cutoff=60; expect=10; Matrix BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

The present description also provides a method for isolating the pluripotent stem cells of the present description. In one aspect, a human placenta is first obtained following a delivery. The cells are then extracted from the placenta. For example, the cells could be enzymatically extracted using trypsin and DNase I (e.g. approximately 35 to 40 g of placental villous tissue is incubated with the enzymes at 37° C.). The cells extracted from the tissue present in the supernatant are then filtered through a nylon mesh and centrifuged in order to obtain a pellet. The resuspended pellet is next layered over a discontinuous Percoll density gradient (5-70%) and centrifuged at 400×g at room temperature for 20 minutes. The cell band between 40 and 50% is collected, washed and cultured. Once the cells are extracted from the placenta, cells positive for human leucocyte antigen-G (HLA-G), a migration marker and at least one pluripotent stem cell marker are isolated. Techniques for isolating cells positive for selective markers are known in the art such as Magnetic Activated Cell Sorting (MACSorting) and/or by Fluorescent Activated Cell Sorting (FACSorting). As known these methods rely on antibodies capable of binding to a selected marker. The cells being positive for the marker are then sorted from the cells that do not express the marker.

As described in the Examples, the pluripotent stem cells of the present description were isolated using MACSorting. By using the MACSorting technique, 18.75% of the cells isolated are positive for CXCR4, HLA-G and SSEA4. As also described in the Examples, the pluripotent stem cells of the present description were also isolated using the MACSorting technique to isolate cells expressing the migration marker (CXCR4) and by further isolating cells expressing HLA-G and pluripotent stem cell marker (SSEA4, NANOG, ALP or OCT4) using FACSorting technique. By using these 2 techniques combined, 20.25% of the cells isolated are positive for CXCR4, HLA-G and SSEA4. Although the use of MACSorting technique combined to the FACSorting technique allows a better cell recovery than when using MACSorting alone, both techniques could be used independently.

The method of the present description also provides pluripotent stem cells positive for HLA-G, a migration marker and at least one pluripotent stem cell marker as described above.

The pluripotent stem cells of the present description could be used for therapeutic purposes in a wide variety of applications such as regenerative medicine, drug delivery and gene therapy. The pluripotent stem cells of the present description could also be used for medical imaging, cell cosmetics, toxicity testing and as a research tool.

The expression “therapeutic purposes” refers to using the placental stem cells of the present description to treat patient suffering from conditions that involve regenerative medicine.

Regenerative Medicine

The expression “regenerative medicine” refers to a process of replacing or regenerating human cells, tissues or organs to restore or establish normal function. Examples of regenerative medicine include the injection of stem cells, the induction of regeneration by biologically active molecule administered by infused cells, and transplantation of in vitro grown organs and tissues (tissue engineering).

As the pluripotent stem cells of the present description are positive for HLA-G and for a migration marker, they could be used to regenerate a tissue in vivo upon local or systemic administration. In addition, the pluripotent stem cells of the present description could be used for regenerating a tissue in vitro by tissue-engineering. As the stem cells of the present description are positive for HLA-G, the engineered tissue could be grafted without promoting an adverse immune response.

Gene Therapy

The expression “gene therapy” refers to use of DNA as a pharmaceutical agent to treat a disease. The pluripotent stem cells of the present description could be genetically engineered ex vivo with a nucleic acid encoding a specific protein and then be provided to a patient as known in the art. For instance, the cells could be engineered using saponins, cationic polyamines, liposome, microcapsulation, or viruses.

Drug Discovery and Delivery

The expression “drug discovery” refers to the process by which a new candidate drug compound is discovered. The Stem Cell Drug Discovery Approach aims to identify candidates that modulate stem cell function which can demonstrate potential clinical applications. Three specific areas could be concerned: compound identification, compound validation, and/or compound optimization. The cells of the present description could be used for such compound identification, validation and/or optimization. This technology may be applicable in the identification of new drug-like molecules which could further be developed to address unmet medical needs.

The expression “drug delivery” refers to a method of administering a pharmaceutical compound to achieve a therapeutic effect. The cells of the present description could therefore be genetically engineered ex vivo as mentioned above in order to express a particular polypeptide or a particular compound that would have a therapeutic effect to the patient upon delivery. This technology may be applicable to every diseases characterized by absence or insufficiency in the synthesis of a protein, enzyme, hormone, or other components vital to the functioning of the human body. Non limiting examples include: cancer, Alzheimer, Parkinson, diabetes and every diseases resulting from a genetic defect.

Medical Imaging

The cells of the present description could also be used in medical imaging. The latter refers to a process used to create image of the body or parts thereof for clinical purposes to diagnose or study diseases. In one aspect, upon labeling the pluripotent stem cells of the present description, the cells could be tracked by different techniques known in the art. The cells could therefore be tracked and studied within the body. For example, the cells could be labeled with gold particle, a fluorescent molecule or iron and could be detected by MRI, CT-scan, or Xenogen.

Cell Therapy

The expression “cell therapy” refers to the process of introducing new cells into a patient in order to treat a disease. This expression is a sub-type therapy of regenerative medicine. Cell therapy often focuses on the treatment of hereditary disease, with or without the addition of gene therapy. As known, cell therapy is used for many clinical indications, in multiple organs and by several modes of cell delivery. Accordingly, the specific mechanisms of action involved in the therapies are wide ranging. Without being bound to a particular theory, the inventor believes that there are two main known mechanisms by which cells promote therapeutic action:

First, it is known that stem cells differentiate into a specific cell type in vitro as well as upon reaching the site of injury (via local or systemic administration). These cells then integrate into the site of injury replacing damaged tissue improving therefore the function of the organ or tissue. For example, stem cells are known to be used to replace cardiomyocytes after myocardial infarction (Jackson, K. A., S. M. Majka, et al. (2001). “Regeneration of ischemic cardiac muscle and vascular endothelium by adult stem cells.” J Clin Invest 107(11): 1395-402 and Kawada, H., J. Fujita, et al. (2004). “Nonhematopoietic mesenchymal stem cells can be mobilized and differentiate into cardiomyocytes after myocardial infarction.” Blood 104(12): 3581-7).

Second, stem cells have the capacity to release soluble factors such as cytokines, chemokines, and growth factors which act in a paracrine or endocrine manner. These factors facilitate self-healing of the organ or region. The delivered cells (via local or systemic administration) remain viable for a relatively short period (days-weeks) and then die. This includes stem cells that naturally secrete the relevant therapeutic factors, or which undergo epigenetic changes or genetic engineering (gene therapy) that causes the cells to release large quantities of a specific molecule such as cells secreting factors which facilitate angiogenesis, anti-inflammation, and anti-apoptosis (Deuse, T., C. Peter, et al. (2009). “Hepatocyte growth factor or vascular endothelial growth factor gene transfer maximizes mesenchymal stem cell-based myocardial salvage after acute myocardial infarction.” Circulation 120(11 Suppl): S247-54, Kelly, M. L., M. Wang, et al. “TNF receptor 2, not TNF receptor 1, enhances mesenchymal stem cell-mediated cardiac protection following acute ischemia.” Shock 33(6): 602-7, Yagi, H., A. Soto-Gutierrez, et al. “Mesenchymal stem cells: Mechanisms of immunomodulation and homing.” Cell Transplant 19(6): 667-79).

Clinical studies show that stem cells can be injected locally or systemically into a patient resulting in promising cell therapy to restore damage tissue such as brain injury, spinal cord injury, traumatic brain and ischaemic cardiomyopathy (Bulte J W In vivo MRI cell tracking: clinical studies, AJR Am J Roentgenol, 2009, p. 314-25, Bolli R et al. Cardiac stem cells in patients with ischaemic cardiomyopathy (SCIPIO): initial results of a randomized phase 1 trial, The Lancet, 2011, p. 1847-1857).

As such, the pluripotent stem cells of the present description could be used to treat diseases that could be treated by cell therapy such as cardiovascular damage, diabetes, cancer (leukemia, multiple myeloma, lymphoma, glioblastoma or breast cancer), brain damage (caused by stroke or traumatic brain), brain degeneration (Parkinson or Alzheimer), spinal cord injury, amyotrophic lateral sclerosis, wound healing, infertility, crohn's disease or cornea damage.

The present description also provides the use of pluripotent stem cells positive for human leucocyte antigen-G (HLA-G), a migration marker and at least one pluripotent stem cell marker as described above and could be adapted for local or systemic administration.

The expression “systemic administration” refers to a route of administration that is either enteral or parental. Enteral administration is known to involve absorption of the component through the gastrointestinal tract. Parental refers to administration outside the digestive tract and for instance intravenous injection. When the cells of the present description are enterally administered, they may be inserted into dendrimers, lipoprotein-based drug carriers polymeric or micelles as known in the art.

The expression “local administration” refers to application of the cells to a localized area of the body or to the surface of a body part. As such, if the injured site is within the brain, the cells of the present description could be applied in the brain at the injured site.

The present description also provides a cell therapy method for treating a patient in need thereof.

The expression “treating a patient in need thereof” refers to any person susceptible of suffering or suffering from a disease from which the symptoms could be alleviated or reduced. More specifically, the subject consists of a human.

As mentioned above, cell therapy using stem cells could alleviate symptoms caused by diseases such as cardiovascular damage, diabetes, cancer (leukemia, multiple myeloma, lymphoma, glioblastoma or breast cancer), brain damage (caused by stroke or traumatic brain), brain degeneration (Parkinson or Alzheimer), spinal cord injury, amyotrophic lateral sclerosis, wound healing, infertility, crohn's disease, or cornea damage. As such, a local or a systemic administration of stem cells to a patient susceptible or suffering from said diseases could alleviate the symptoms.

The method of the present description also provides pluripotent stem cells positive for human leucocyte antigen-G (HLA-G), a migration marker and at least one pluripotent stem cell marker as described above.

The pluripotent stem cells of the present description positive for HLG-A are immuno tolerant and could be immunocompatible with any receiver without risking a graft rejection. As such, the cells of the present description isolated from human could be injected to any animals without risking a graft rejection. In addition, the pluripotent stem cell of the present description positive for a migration marker could migrate to the target such as an injured site. In addition, the pluripotent stem cells of the present description are isolated from placenta which requires no manipulation of an embryo. As such, no ethical issue is raised upon isolating the pluripotent stem cells of the present description. In one aspect, the placenta allows the recovery of a large quantity of stem cells.

Cell Cosmetics

The pluripotent stem cells of the present description could be used in cell cosmetics such as Placental Stem Cell Cosmetics (PSCC) or Placental Stem Cell Cosmeceuticals (PSCC). The expression “cell cosmetic” refers to cosmeceuticals based on cells extracted from organs or tissues. Known cell cosmeceuticals are based on biochemical substances isolated from embryo tissues or placenta. The pluripotent stem cells of the present description provide therefore a source of placental stem cells for cosmeceuticals treating disorders affecting skin such as frostbite, burn, skin loss, skin ulcer, gangrene, slough, dermatitis, eczema, acne, psoriasis and zona. In addition, the pluripotent stem cells of the present description could be used in cosmetics for women, men and babies.

Research Tools

The pluripotent stem cells of the present description could be used as a research tool such as a model for human genetic disorders or as a model for toxicity testing.

Model for Human Genetic Disorders

The pluripotent stem cells of the present description could be used as a model for human genetic disorder by genetically manipulating the cells or by deriving diseased cell lines. This approach could be useful for studying disorders such as fragile-X syndrome, cystic fibrosis, trisomy, and other genetic disorders that have no reliable study model.

Model for Toxicity Testing

The pluripotent stem cells of the present description allow the development of a novel toxicity test platform to accelerate drug development and identify new toxic factors. This new toxicity test platform allows an alternative to animal tests. The pluripotent stem cells of the present description further allow the development of toxicity tests using pluripotent stem cells lines subjected to standardised culture and differentiation protocols. The different tests cover reproductive toxicity, neurotoxicity, metabolism and toxicokinetics, and could finally be integrated into an “all-in-one” test system. Pluripotent stem cells culture could be automated and scaled-up to enable future industrial use of the developed toxicity tests.

The cells of the present description could be administered to a patient by mixing an effective amount of cells and optionally other active substance with a pharmaceutically acceptable carrier such as diluent, excipient or a vehicle. The carrier could have different forms depending on the route of administration as known in the art.

It will be appreciated that the amount of cells to be injected could vary with the nature of the condition for which treatment is sought and the condition of the patient and could be ultimately at the discretion of the attending physician. The quantity of the cells of the present description to be administered in a patient could be at least 2×10³/kg; about 2×10³/kg to 3×10⁶/kg, about 2×10³/kg to 1.6×10⁴/kg, about 5×10⁴/kg to 5×10⁵/kg or about 6×10⁵/kg to 3×10⁶/kg (see for e.g. Kebriaei P et al. (2009). “Adult human mesenchymal stem cells added to corticosteroid therapy for the treatment of acute graft-versus-host disease” Biol Blood Marrow Transplant 15(7): 804-11, Björklund L M et al, (2002) “Embryonic stem cells develop into functional dopaminergic neurons after transplantation in a Parkinson rat model” PNAS, 99(4): 2344-2349).

The present description will be more readily understood by referring to the following examples. The scope of the claims should not be limited by the preferred embodiments set forth in the examples, but should be given the broadest interpretation consistent with the description as a whole.

### EXAMPLES

### Example 1

Upon extraction of the cells, a MACSorting is performed and then all three phenotypes are sorted. As shown in FIG. 1 and in table 1, 44.25% of the cells isolated are positive for SSEA4, 29.03% of the cells are positive for HLA-G and 75.25% are positive for CXCR4. 18.75% of the isolated cells are positive for SSEA4, HLA-G and CXCR4.

TABLE 1

| | SSEA4 | HLA-G | CXCR4 | ALL |
|---|---|---|---|---|
| Number of values | 4 | 4 | 4 | 4 |
| Minimum | 33.00 | 24.00 | 64.00 | 15.00 |
| 25% Percentile | 34.25 | 24.78 | 66.00 | 15.50 |
| Median | 44.50 | 28.57 | 76.00 | 18.50 |
| 75% Percentile | 54.00 | 33.75 | 83.75 | 22.25 |
| Maximum | 55.00 | 35.00 | 85.00 | 23.00 |
| 10% Percentile | 33.00 | 24.00 | 64.00 | 15.00 |
| 90% Percentile | 55.00 | 35.00 | 85.00 | 23.00 |
| Mean | 44.25 | 29.03 | 75.25 | 18.75 |
| Std. Deviation | 10.44 | 4.672 | 9.215 | 3.500 |
| Std. Error | 5.218 | 2.336 | 4.608 | 1.750 |

### Example 2

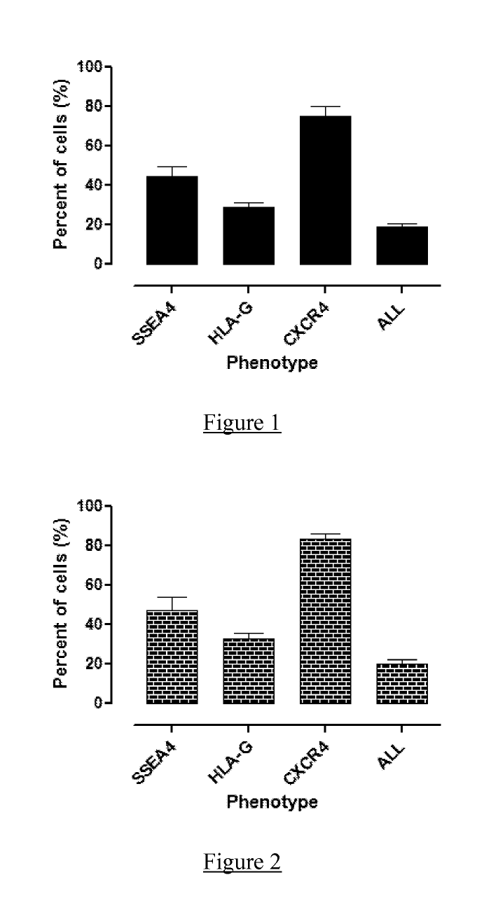

Upon extraction of the cells, a MACSorting is performed to isolate cells positive for CXCR4 and a FACSorting is further performed with the cells positive for CXCR4 to isolate cells positive for SSEA4 and HLA-G. As shown in FIG. 2 and in table 2, 47.04% of the isolated cells are positive for SSEA4, 32.75% are positive for HLA-G and 83.50% are positive for CXCR4. 20.25% of the isolated cells are positive for SSEA4, HLA-G and CXCR4.

TABLE 2

| | SSEA4 | HLA-G | CXCR4 | ALL |
|---|---|---|---|---|
| Number of values | 4 | 4 | 4 | 4 |
| Minimum | 30.77 | 28.00 | 79.00 | 17.00 |
| 25% Percentile | 33.58 | 28.25 | 79.25 | 17.25 |
| Median | 48.50 | 31.00 | 82.50 | 19.50 |
| 75% Percentile | 59.04 | 39.00 | 88.75 | 24.00 |
| Maximum | 60.38 | 41.00 | 90.00 | 25.00 |
| Mean | 47.04 | 32.75 | 83.50 | 20.25 |
| Std. Deviation | 13.31 | 5.909 | 5.066 | 3.594 |
| Std. Error | 6.655 | 2.955 | 2.533 | 1.797 |
| Lower 95% CI of mean | 25.86 | 23.35 | 75.44 | 14.53 |
| Upper 95% CI of mean | 68.22 | 42.15 | 91.56 | 25.97 |

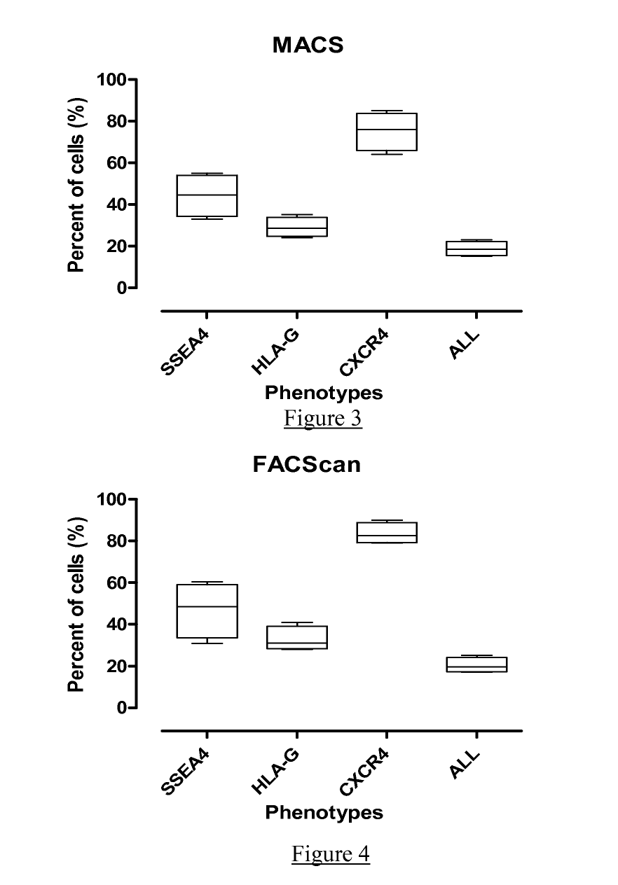

FIGS. 3 and 4 show the comparison between the percentages of cells obtained using the MACSorting technique versus the FACSorting technique.

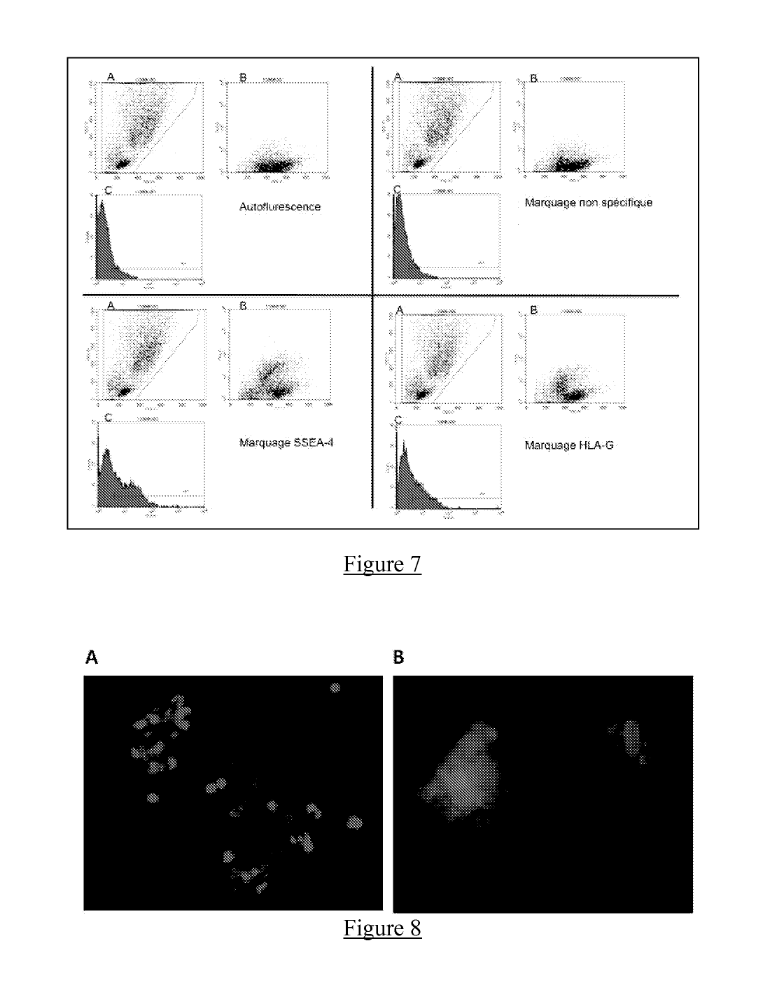

FIG. 7 represents an illustration of FACScan analysis confirming the presence of SSEA4 or HLA-G.

### Example 3

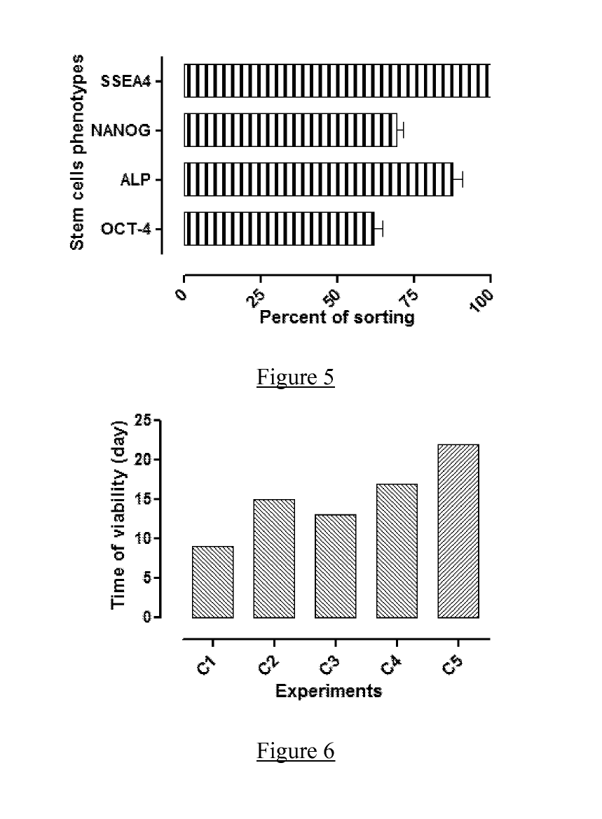

Stem cells positive for SSEA4 were further sorted by MACSorting in order to determine if they are also positive for other pluripotent stem cell such as NANOG, ALP and OCT-4. FIG. 5 shows that these cells are also positive for NANOG, ALP and OCT-4. Table 3 shows that 69.50%, 87.50% and 62% of the cells positive for SSEA4 are further positive for NANOG, ALP and OCT-4, respectively.

TABLE 3

| | SSEA4 | NANOG | ALP | OCT-4 |
|---|---|---|---|---|
| Number of values | 4 | 4 | 4 | 4 |
| Minimum | 100.0 | 65.00 | 80.00 | 55.00 |
| 25% Percentile | 100.0 | 65.75 | 81.25 | 56.25 |
| Median | 100.0 | 69.00 | 87.50 | 62.50 |
| 75% Percentile | 100.0 | 73.75 | 93.75 | 67.25 |
| Maximum | 100.0 | 75.00 | 95.00 | 68.00 |
| Mean | 100.0 | 69.50 | 87.50 | 62.00 |
| Std. Deviation | 0.0 | 4.203 | 6.455 | 5.715 |
| Std. Error | 0.0 | 2.102 | 3.227 | 2.858 |
| Lower 95% CI of mean | 100.0 | 62.81 | 77.23 | 52.91 |
| Upper 95% CI of mean | 100.0 | 76.19 | 97.77 | 71.09 |

### Example 4

The in vitro viability of the isolated cells was also investigated. As shown in FIG. 6, cells could survive up to 22 days in culture.

### Example 5

FIG. 8 shows a representative CXCR4 immunostaining of the stem cells of the present description. A higher immunofluorescence intensity (score 3) is detected in stem cells positive for CXCR4, HLA-G and SSEA4 (B) than in negative control stem cells (score 1) (A).

### Example 6

Isolated stem cells of the present description do not cause graft-vs-host disease, teratomas or tumors when injected into the tail vein, subcutaneously, intramuscularly or intraperitoneally in immunodepressed Nude mice during four weeks (Table 4). These results confirm the readiness of the population of stem cells to be used in regenerative medicine.

TABLE 4

| | Cell transplantation | | |
|---|---|---|---|
| Diseases | protocol | Outcome | Status |
| Acute graft-vs- | Intravenous injection | Diarrhea | Not occurred |
| host disease | | Weight loss | Not occurred |
| | | Skin changes | Not occurred |
| | | Distress | Not occurred |
| | | Death | Not occurred |
| Teratoma | Subcutaneous injection | Teratoma | Not found |
| formation | Intramuscular injection | | |
| Tumor formation | Intravenous injection | Tumors | Not found |
| | Intraperitoneal injection | | |

Materials and Methods

Human Subjects

Placentas are obtained from women with normal pregnancy and delivery, and all neonates are of appropriate size for gestational age. Eligible participants are not known for suffering from any medical conditions (i.e. diabetes, hypertension or metabolic disease) nor cigarette, alcohol or illicit drug use. All participants give their written and informed consent to take part of the study, which is approved by the CHUS Ethics Human Research Committee on Clinical Research.

Collection of Placental Samples

Immediately after delivery of the placenta, samples are collected, and the total length of processing time is less than 15 min. Tissue of the placenta is taken avoiding areas of infarcts and thrombosis.

Stem Cells Isolation and Culture

Placental cells are purified from placentas of uncomplicated term pregnancies immediately after delivery according to our previous study (Aris A. et al. Detrimental effects of high levels of antioxidant vitamins C and E on placental function: considerations for the vitamins in preeclampsia (VIP) trial J. Obstet. Gynaecol. Res. 2008, p. 504-511 and Benachour N. et al. Toxic effects of low doses of bisphenol-A on human placental cells Toxicol Appl Pharmacol 2009, p. 322-8). Typically, 35 to 40 g of placental villous tissue is digested three times for 30 min with 0.15% Trypsin and 0.02% DNase I in HBSS containing 25 mM HEPES in a water bath at 37° C. For the two last digestions, tissue fragments are allowed to settle and the supernatant is filtered through a 200 μM pore size nylon mesh and no more than 45 ml of cell suspension is layered over 5 ml aliquots of FCS serum and centrifuged at 1000×g for 10 min at room temperature. The pelleted cells are then resuspended in DMEM at room temperature. All of the resultant cell suspensions are pooled, centrifuged at 1000×g, and resuspended in 5 ml of DMEM. The cells are layered over a discontinuous Percoll density gradient (5-70% in 5% steps of 3 ml each in HBSS 1×) and centrifuged at 400×g at room temperature for 20 min. The cell bands between 40% and 50% is collected, washed once, and cultured. Various media may be used for culturing these cells, such as DMEM, F-12, M199, RPMI, Fisher's, Iscore's, McCoy's and their combinations. These media may be supplemented with fetal bovine serum (FBS), whole human serum (WHS), or human umbilical cord serum collected at the time of delivery of the placenta. To this is added 1% PSN, 2 mM glutamine and 44 mM sodium bicarbonate at pH 7.2 in a humidified 5% CO₂/95% air incubator at 37° C.

In order to avoid interference of FBS, cells could be washed with media (i.e. DMEM) without FBS, and then washed with physiological saline.

Magnetic Activated Cell Sorting (MACSorting)

Twenty billions cells are washed twice with MACS buffer (PBS, 0.5% BSA and 2 mM EDTA). Cells are than incubated with primary antibodies (Tables 5 and 6) during 35 minutes at 4° C. in MACS buffer. After this step, cells are washed twice in 2 ml of the same buffer and incubated during 17 minutes at 4° C. with antimouse IgG MicroBead (Milteny biotech) according to the manufacturer's instructions. Cells are washed twice with cold buffer and then suspended in 1 ml of cold buffer. An Ms column (Milteny biotech) is conditioned with 500 μl of cold buffer and then the labeled cells are allowed to pass through the column. The column is washed with 3×500 μl of cold buffer and then demagnetized and eluted with 1 ml of cold buffer by gravity and with 1 ml of cold buffer by pushing the piston to force the buffer through the column according to the manufacturer's instructions.

When another MACS is performed on the same cells, these cells should return in culture for at least 4 hours, allowing them to internalize and digest the previous antibodies. These cells are then washed twice and suspended in 500 μl of cold buffer. A new column was conditioned for the passage of cells in suspension. The column is washed twice with 2 ml of cold buffer. Repeat this process according to the number of required sorting.

TABLE 5

| Antibody | Manufacturer | Used concentration |
|---|---|---|
| Ms mAb Anti h/m SSEA-4 | R&D System | 2.5 μg/ml |
| Ms mAb HLA-G [MEMG/9] | Abcam | 2.5 μg/ml |
| Ms mAb CXCR4 | Abcam | 2.5 μg/ml |

TABLE 6

| Antibody | Manufacturer | Used concentration |
|---|---|---|
| Ms mAb NANOG [NNG-811] | Abcam | 2.5 μg/ml |
| Ms mAb Alkaline | Abcam | 2.5 μg/ml |
| Phosphatase | | |
| [BGN/03/661] | | |
| Ms mAb OCT-4 | Abcam | 2.5 μg/ml |

Fluorescent Activating Cell Sorting (FACSorting)

After a MACS directed against CXCR4, cells are washed twice with FACSorting buffer (PBS Ca/Mg⁺⁺ Free, 25 mM HEPES, pH 7 and 1% FBS which is heat inactivated and sterilized by filtration). Cells are then incubated with conjugated antibodies (Table 7) during 30 minutes at 4° C. in the dark. Cells are then washed twice with cold buffer and filtered with a 60 μm nylon mesh filter. Finally, the suspended cells are sorted using a FACSAria III cell sorter and collected in 5 ml tube and returned to culture.

TABLE 7

| Antibody | Manufacturer | Used concentration |
|---|---|---|
| Ms mAb SSEA-4 PE | R&D System | 0.25 μg/1 000 000 ¢ |
| Ms mAb HLA-G [MEMG/9]- | Thermo Scientific | 1 μg/ml |
| APC | | |
| Ms mAb CXCR4 -PE-Cy ™5 | BD Pharmingen | ⅙ dilution |

PE: phycoerythrin, APC: allophycocyanin

Cells Viability and Counting

Cell viability depends on the confluence. In general, a confluence under 5% ends the experiment.

Blue Trypan Counting

A manual count using hemocytometer. Viability is evaluated by trypan blue exclusion method.

Fluorescent Activating Cell Scan (FACScan)

150 000 freshly isolated cells are washed twice in a FACScan buffer containing PBS and 0.5% BSA. Cells are then incubated with primary antibody (Table 7) during 30 minutes at 4° C. Cells are then washed twice with co Id buffer and incubated with goat anti-mouse IgG conjugated with phycoerythrin (R&D System) in cold buffer during 30 minutes on ice in the dark. After twice washes, cells are suspended in 200 μl of the same buffer and kept in the dark until the analysis, which must be done within one hour. A tube without the primary antibody and a tube with only the secondary antibody could be used as controls.

Immunostaining

The expression of CXCR4 was assessed by immunofluorescence. Briefly, adhered stem cells were fixed in 3.7% of paraformaldehyde (Paraformaldehyde 37%, Sigma Aldrich, USA) for 20 minutes at room temperature. After permeabilized for 4 minutes in PBS—Triton X100 0.3%, cells were incubated for 30 min in PBS—10% goat serum—1% BSA (Bovine Serum Albumin, Sigma, USA)—Triton X100 0.1% to reduce the non-specific binding. Cells were then incubated overnight with 3 μg/ml of monoclonal anti-CXCR4 (Ms mAb to CXCR4, Abcam, USA) in the blocking buffer. Then, cells were incubated for 1 hour with Alexa fluor 594 anti-mouse IgG (Invitrogen Life Sciences, USA). Controls samples included reactions where the primary antibody was omitted or where the primary antibody was replaced with corresponding matched negative control immunoglobulins at the same concentration as the primary antibody. Then, cells were washed and counterstained with diamidino-2-phenylindole for (DAPI) for 10 minutes at room temperature. Following mounting, cells were viewed using the Olympus BX61 microscope and photomicrographs were taken with a corporate computer.

Immune Tolerance and Safety

Two endpoints were investigated using a mouse model: 1) development of acute graft-vs-host disease (aGVHD), and 2) teratoma and tumors formation. Four 8-10-wk-old males NU/NU Nude mice were used (Charles River, Canada).

Following isolation, the cells as described herein could be further treated to isolate the antibodies used for isolation from said cells. In one embodiment, the isolated cells are treated with about 0.25% trypsin-EDTA for about 5 minutes at about 37° C. such that the antibodies used for isolation could be separated from said cells.

1) Development of aGVHD

Isolated cells (5×10⁵) suspended in PBS were injected into the tail vein and intraperitoneally in two immunodepressed nude mice. Mice were monitored daily for four weeks for aGVHD clinical signs including: diarrhea, weight loss, skin changes, respiratory distress or sudden death related to acute pulmonary edema (Filipovich A H et al. National Institutes of Health Consensus Development Project on Criteria for Clinical Trials in Chronic Graft-versus-Host Disease: I. Diagnosis and Staging Working Group Report. Biology of Blood and Marrow Transplantation. 2005 December, 11(12):945-956).

2) Teratoma and Tumors Formation

Isolated cells (5×10⁵) suspended in PBS were injected subcutaneously or intramuscularly into the leg of two immunodepressed Nude mice. Animals were sacrificed 4 weeks post-transplantation, and the injection sites were visually inspected for teratoma and tumors formation.

1-48. (canceled)

49. An isolated population of human placental pluripotent stem cells or an isolated human placental pluripotent stem cell, positive for human leucocyte antigen-G (HLA-G); a migration marker chosen from chemokine receptor CXCR4, CXCR5, CXCR6, CXCR7, CCR1, CCR2, CCR3, CCR4, CCR7, CCR9, platelet-derived growth factor receptor (PDGF-Rα), PDGF-Rβ, insulin-like growth factor receptor (IGF-R), RANTES-R and MDC-R; and a pluripotent stem cell marker that is SSEA4.

50. The isolated population of human placental pluripotent stem cells or the isolated human placental pluripotent stem cell according to claim 49, wherein the migration marker is CXCR4.

51. An isolated population of human placental pluripotent stem cells or an isolated human placental pluripotent stem cell, positive for human leucocyte antigen-G (HLA-G), a migration marker and at least one pluripotent stem cell marker.

52. The isolated population of human placental pluripotent stem cells or the isolated human placental pluripotent stem cell of claim 51, wherein the at least one pluripotent stem cell marker is stage-specific embryonic antigen SSEA4, SSEA3, POU5F1/OCT4, NANOG, SOX2, alkaline phosphatase (ALP), human embryonic stem cell antigen-1 (HESCA-1), developmental pluripotency associated 5 (DPPA5), forkhead box D3 (GENESIS/FOXD3), undifferentiated embryonic cell transcription factor 1 (UTF1), TRA-1-60, TRA-1-81, DNA (cytosine-5-)-methyltransferase 3 beta (DNMT3B), teratocarcinoma-derived growth factor 1 (TDGF1/CRIPTO), reduced expression gene 1 (REX1/ZFP42), telomerase reverse transcriptase (TERT), ATP-binding cassette sub-family G member 2 (ABCG2), connexin-43, connexin-45, GCTM2, GCT343, thymus cell antigen (Thy1/CD90), gamma-aminobutyric acid receptor subunit beta-3 (GABRB3), CD9, growth differentiation factor-3 (GDF3), STELLAR, or fibroblast growth factor 4 (FGF4).

53. The isolated population of human placental pluripotent stem cells or the isolated human placental pluripotent stem cell of claim 51, wherein the at least one pluripotent stem cell marker is SSEA4, NANOG, ALP or OCT4.

54. The isolated population of human placental pluripotent stem cells or the isolated human placental pluripotent stem cell according to claim 51, wherein the isolated population or isolated cell are positive for pluripotent stem cell markers SSEA4, NANOG, ALP and OCT4.

55. The isolated population of human placental pluripotent stem cells or the isolated human placental pluripotent stem cell according to claim 51, wherein the pluripotent stem cell marker is SSEA4.

56. The isolated population of human placental pluripotent stem cells or the isolated human placental pluripotent stem cell according to claim 51, wherein the migration marker is chemokine receptor CXCR 4, CXCR5, CXCR6, CXCR7, CCR1, CCR2, CCR3, CCR4, CCR7, CCR9, platelet-derived growth factor receptor (PDGF-Rα), PDGF-Rβ, insulin-like growth factor receptor (IGF-R), RANTES-R, or MDC-R.

57. The isolated population of human placental pluripotent stem cells or the isolated human placental pluripotent stem cell according to claim 51, wherein the migration marker is CXCR4.

58. A cell therapy method for treating a patient in need thereof, the method comprising administering to the patient cells obtained from the isolated population of human placental pluripotent stem cells or the isolated human placental pluripotent stem cell according to claim 49.

59. A cell therapy method for treating a patient in need thereof, the method comprising administering to the patient cells obtained from the isolated population of human placental pluripotent stem cells or the isolated human placental pluripotent stem cell according to claim 50.

60. A cell therapy method for treating a patient in need thereof, the method comprising administering to the patient cells obtained from the isolated population of human placental pluripotent stem cells or the isolated human placental pluripotent stem cell according to claim 51.

61. The cell therapy method of claim 50, wherein the administration is a local or a systemic administration.

62. The cell therapy method of claim 60, comprising treating cardiovascular damage, diabetes, cancer, brain damage, brain degeneration, spinal cord injury, amyotrophic lateral sclerosis, wound healing, infertility, crohn's disease, or cornea damage.

63. A method for isolating human placental pluripotent stem cells, the method comprising:
extracting cells from a human placenta;
incubating extracted cells with a first maker primary antibody specific for a first marker selected from human leucocyte antigen-G (HLA-G), a migration marker and at least one pluripotent stem cell marker;
washing said incubated extracted cells with a first buffer;
incubating said washed extracted cells with a secondary antibody that is specific for said first marker primary antibody;
isolating cells positive for said first marker;
optionally culturing said isolated cells positive for said first marker;
incubating said isolated cells positive for said first maker with a second marker primary antibody specific for a second marker selected from human leucocyte antigen-G (HLA-G), a migration marker and at least one pluripotent stem cell marker, wherein said second marker and said first marker are different markers;
washing said incubated extracted cells with a second buffer;
incubating said washed extracted cells with a secondary antibody that is specific for said marker primary antibody;
isolating cells positive for said second marker;
optionally culturing said isolated cells positive for said second marker;
incubating said isolated cells positive for said second maker with a third marker primary antibody specific for a third marker selected from human leucocyte antigen-G (HLA-G), a migration marker and at least one pluripotent stem cell marker, wherein said third marker, said second marker and said first markers are each different markers;
washing said incubated extracted cells with a third buffer;
incubating said washed extracted cells with a secondary antibody that is specific for said third marker primary antibody;
isolating cells positive for said third marker;
thereby isolating cells positive for human leucocyte antigen-G (HLA-G), a migration marker and at least one pluripotent stem cell marker.

64. The method of claim 63, wherein the at least one pluripotent stem cell marker is stage-specific embryonic antigen SSEA4, SSEA3, POU5F1/OCT4, NANOG, SOX2, alkaline phosphatase (ALP), human embryonic stem cell antigen-1 (HESCA-1), developmental pluripotency associated 5 (DPPA5), forkhead box D3 (GENESIS/FOXD3), undifferentiated embryonic cell transcription factor 1 (UTF1), TRA-1-60, TRA-1-81, DNA (cytosine-5-)-methyltransferase 3 beta (DNMT3B), teratocarcinoma-derived growth factor 1 (TDGF1/CRIPTO), reduced expression gene 1 (REX1/ZFP42), telomerase reverse transcriptase (TERT), ATP-binding cassette sub-family G member 2 (ABCG2), connexin-43, connexin-45, GCTM2, GCT343, thymus cell antigen (Thy1/CD90), gamma-aminobutyric acid receptor subunit beta-3 (GABRB3), CD9, growth differentiation factor-3 (GDF3), STELLAR, or fibroblast growth factor 4 (FGF4).

65. The method of claim 64, wherein the pluripotent stem cell marker is SSEA4.

66. The method of claim 65, wherein the migration marker is CXCR4.

67. The method of claim 63, wherein said step of isolating cells positive for said first marker is performed by magnetic activated sorting; said step of isolating cells positive for said second marker is performed by magnetic activated sorting; and said step of isolating cells positive for said third marker is performed by fluorescent activating cell sorting.

68. The method of claim 63, wherein said isolated cells are positive for CXCR4, SSEA4 and HLA-G.
